(19) United States
(12) Patent Application Publication
(10) Pub. No.: US 2026/0219141 A1
(43) Pub. Date: Jul. 30, 2026

(54) METHOD AND SYSTEM FOR MEASURING EMISSION RATE OF AIRBORNE EMISSION SPECIES

(71) Applicant: AEROMON OY, Helsinki (FI)

(72) Inventors: Matti IRJALA, Helsinki (FI); Jouko SALO, Helsinki (FI)

(21) Appl. No.: 19/142,683

(22) PCT Filed: Dec. 29, 2023
(86) PCT No.: PCT/FI2023/050748

§ 371 Date: Jun. 24, 2025

(30) Foreign Application Priority Data
Dec. 30, 2022 (FI) 20226191

(51) Int. Cl. G01N 1/22; B64U 101/35; G01N 33/00; G05D 1/646; G05D 105/80; G05D 107/70; G05D 109/25

(52) U.S. Cl. CPC G01N 1/2273; G01N 33/0004; G05D 1/646; B64U 2101/35; G05D 2105/80; G05D 2107/70; G05D 2109/254

(57) ABSTRACT

A method and system for measuring emissions rate of airborne emission species from an emission site includes releasing tracer gas species from a tracer gas source at a determined tracer gas mass flow rate, providing an unmanned aerial vehicle, controlling the unmanned aerial vehicle to flight along a first flight trajectory, measuring at different altitudes concentration of at least one airborne emission species emitted from the emission site, and concentration the tracer gas species emitted from the tracer gas source. The method further includes determining emission rate of the at least one airborne emission species from the emission site at the different altitudes of the first flight trajectory.

### FIELD OF THE INVENTION

The present invention relates to a method for measuring emission rate of airborne emission species and more particularly to a method according to preamble of claim 1. The present invention further relates to a system for measuring emission rate of airborne emission species and more particularly to a system according to preamble of claim 12.

### BACKGROUND OF THE INVENTION

There are several different methods for determination of airborne emissions from emissions sites. One of the used methods is so called tracer gas method. The tracer gas method is a remote sensing method used for quantifying airborne emissions. The tracer gas method combines a controlled release of tracer gas from the emission site with concentration measurements downwind of the emission site, by using analytical instruments. The tracer gas method in general is based on the assumption that a tracer gas, not too dissimilar to the target compound, released at the emission site will disperse in the atmosphere in the same way as airborne emissions emitted from the emission site. Assuming a defined wind direction, well mixed air with airborne emissions and tracer gas, and a constant tracer gas release, the airborne emission rate can be calculated as a function of the ratio of concentration of the emitted airborne emissions and concentration of the released tracer gas.

In prior art, the measurements of the airborne emissions from the emission site and the tracer gas an analyser which is incorporated to a car. The measurements are carried out by driving the car along roads in the vicinity of the emission site, preferably downwind direction of the emission site, and simultaneously measuring the airborne emissions and the tracer gas with the analyser provided to the car. Accordingly, tracer gas method is used in prior art applications where emission source locations may be unknown and the sources are relatively small with emission points near ground level.

One of the problems associated with the prior art is that the successful measurements of the tracer gas method need optimal weather conditions for achieving reliable and accurate measurement results. Further, the results from prior art methods maybe inaccurate or difficult due to complex weather conditions carrying the emission in the atmosphere. The emission sites are often in remote areas outside cities with limited or restricted accessibility by car. Therefore, the wind direction needs to be such that the measurements can be carried out along the existing roads. This causes delays and waiting of appropriate wind conditions for carrying out the analysis, as well as somewhat compromised measurement results.

### BRIEF DESCRIPTION OF THE INVENTION

An object of the present invention is to provide method and system for measuring emission rate of airborne emission species so as to solve or at least alleviate the prior art disadvantages.

The objects of the invention are achieved by a method which is characterized by what is stated in the independent claim 1. The objects of the invention are further achieved by a system which is characterized by what is stated in the independent claim 12.

The preferred embodiments of the invention are disclosed in the dependent claims.

The invention is based on the idea of providing a method for measuring emission rates of airborne emission species from an emission site, the emission site emitting one or more airborne emission species. The method comprises:
releasing tracer gas species from a tracer gas source at a determined tracer gas mass flow rate, the tracer gas source being provided to, or in vicinity thereof, the emission site,
providing an unmanned aerial vehicle comprising:
   a control system for controlling flight of the unmanned aerial vehicle, and
   an analyser unit provided to the unmanned aerial vehicle;
controlling the unmanned aerial vehicle with the control system to flight along a first flight trajectory in vicinity of the emission site, the first flight trajectory comprising different altitudes;
measuring at different altitudes with the analyser unit of the unmanned aerial vehicle during the flight of the unmanned aerial vehicle along the first flight trajectory:
   concentration of at least one airborne emission species emitted from the emission site, and
   concentration of the tracer gas species emitted from the tracer gas source; and
determining emission rate of the at least one airborne emission species from the emission site at the different altitudes of the first flight trajectory based on:
   the determined tracer gas mass flow rate of the tracer gas species released from the tracer gas source,
   measured concentration of the tracer gas species emitted from the tracer gas source, and
   measured concentration of the at least one airborne emission species emitted from the emission site.

Determining the emission rate at different altitudes by utilizing the unmanned aerial vehicle enables measuring accurate emission rate from the emission site in any direction in relation to the emission site. Therefore, the emission rate may be determined despite of the wind direction as the movement of the unmanned aerial vehicle is not restricted to existing roads. Further, determining the emission rate from the emission site at different, or at least two altitudes, enables measuring accurate emission rate at different weather conditions when the emissions spread to different altitudes depending on the weather conditions.

In the present application the different altitudes mean different altitudes from the ground surface or water surface.

In some embodiments, the method comprises controlling the unmanned aerial vehicle with the control system along the first flight trajectory, and that the first flight trajectory comprises measurement intervals along the first flight trajectory at different altitudes, the measuring of the concentration of the at least one airborne emission species emitted from the emission site and of the concentration the tracer gas species emitted from the tracer gas source is carried out in the measurement intervals during the flight of the unmanned aerial vehicle.

Accordingly, the concentration measurements may be carried out at different altitudes by utilizing successive measurement intervals.

In some other embodiments, method comprises controlling the unmanned aerial vehicle with the control system along the first flight trajectory, and measuring of the concentration of the at least one airborne emission species emitted from the emission site and of the concentration the tracer gas species emitted from the tracer gas source is carried out continuously during the flight of the unmanned aerial vehicle along the first flight trajectory at different altitudes.

The continuous concentration measurement may be carried out along the first flight trajectory at different altitudes.

In preferably embodiments, sampling time of the concentration measurements is less than 20 s, more preferably less than 10 s. The sampling time means the time interval of concentration measurements generating a discrete measurement sample and further one emission rate output value.

In some embodiments, the method comprises controlling the unmanned aerial vehicle with the control system along the first flight trajectory, the first flight trajectory comprises horizontal flight paths, the horizontal flight paths being at different altitudes, and the measuring of the concentration of the at least one airborne emission species emitted from the emission site and of the concentration the tracer gas species emitted from the tracer gas source is carried along the horizontal flight paths at different altitudes during the flight of the unmanned aerial vehicle.

In some embodiments, the method comprises controlling the unmanned aerial vehicle with the control system along the first flight trajectory, the first flight trajectory comprises horizontal flight paths, the vertical flight paths being at different altitudes, and the measuring of the concentration of the at least one airborne emission species emitted from the emission site and of the concentration the tracer gas species emitted from the tracer gas source is carried along the vertical flight paths at different altitudes during the flight of the unmanned aerial vehicle.

In some embodiments, the method comprises controlling the unmanned aerial vehicle with the control system along the first flight trajectory, the first flight trajectory comprises horizontal and vertical flight paths, the horizontal flight paths being at different altitudes, and the measuring of the concentration of the at least one airborne emission species emitted from the emission site and of the concentration the tracer gas species emitted from the tracer gas source is carried along the horizontal and vertical flight paths, or along the vertical or horizontal flight paths at different altitudes during the flight of the unmanned aerial vehicle.

In some embodiments, the method comprises controlling the unmanned aerial vehicle with the control system along the first flight trajectory, the first flight trajectory comprises circumferential flight paths surrounding the emission site, the circumferential flight paths being at different altitudes, and the measuring of the concentration of the at least one airborne emission species emitted from the emission site and of the concentration the tracer gas species emitted from the tracer gas source is carried along the circumferential flight paths at different altitudes during the flight of the unmanned aerial vehicle.

The circumferential flight paths of the first flight trajectory may be horizontal flight paths. In this case the unmanned aerial vehicle is moved from one horizontal circumferential flight path to another in vertical direction.

Alternatively, the circumferential flight paths are provided a spiral-like flight trajectory in which the length of the circumference decreases as the altitude increases.

In some embodiments, the first flight trajectory preferably is formed in a vertical plane or in a substantially vertical plane.

The first flight trajectory may be predetermined before the flight or measurements of the unmanned aerial vehicle.

In some embodiments, the method comprises determining wind direction in the emission site or in the vicinity of the emission site.

In some other embodiments, the method comprises determining wind direction and wind speed in the emission site or in the vicinity of the emission site.

Wind direction or wind direction and wind speed is utilized for determining direction in which the emission plume spreads from the emission site. Thus, the first flight trajectory may be located to downwind from the emission site.

Accordingly, the vertical plane defined by the first flight trajectory may also be located downwind from the emission site based on the determined wind direction or wind direction and wind speed.

It should be noted that in the context of this invention, the terms vertical and horizontal means directions in relation to ground surface or water surface, or direction is relation to direction of the gravity. Further, it should be noted that terms vertical and horizontal mean directions substantially in vertical or horizontal direction, for example +/−10 degrees to vertical and horizontal directions.

In some embodiments, the method further comprises:
determining location of an emission measurement area in relation to the emission site based on the determined wind direction or based on the determined wind direction and wind speed, the location of the emission measurement area being in downwind direction from the emission site,
controlling the unmanned aerial vehicle with the control system to flight along the first flight trajectory in the vicinity of the emission site in the emission measurement area, and
measuring emissions from the emission site with the analyser unit of the unmanned aerial vehicle during the flight of the unmanned aerial vehicle along the first flight trajectory at different altitudes in the emission measurement area by measuring:
   concentration of at least one airborne emission species emitted from the emission site in the emission measurement area, and
   concentration of the tracer gas species emitted from the tracer gas source in the emission measurement area.

Accordingly, the location of the emission measurement area is determined in relation to the emission site based on the wind direction or based on the wind direction and wind speed. The first flight trajectory or the vertical plane defined by the first flight trajectory is provided to the emission measurement area. Accordingly, the location of the emission measurement area is arranged to define the location of the first flight trajectory or the vertical plane.

In some embodiments, the method further comprises:
determining location of the background measurement area in relation to the emission site based on the determined wind direction or based on the determined wind direction and wind speed, the location of the background measurement area being in upwind direction from the emission site,
controlling the unmanned aerial vehicle with the control system to flight along the first flight trajectory in the vicinity of the emission site in the background measurement area, and
measuring background emissions with the analyser unit of the unmanned aerial vehicle during the flight of the unmanned aerial vehicle along the first flight trajectory at different altitudes in the background measurement area by measuring concentration of at least one background airborne emission species in the background emission area.

In some other embodiments, the method comprises:
determining location of the background measurement area in relation to the emission site based on the determined wind direction or based on the determined wind direction and wind speed, the location of the background measurement area being in upwind direction from the emission site,
controlling the unmanned aerial vehicle with the control system to flight along a second flight trajectory in the vicinity of the emission site in the background measurement area, the second flight trajectory comprising different altitudes, and
measuring background emissions with the analyser unit of the unmanned aerial vehicle during the flight of the unmanned aerial vehicle along the second flight trajectory at different altitudes in the background measurement area by measuring concentration of at least one background airborne emission species in the background emission area.

Accordingly, the location of the background measurement area is determined in relation to the emission site based on the wind direction or based on the wind direction and wind speed. The second flight trajectory or the vertical plane defined by the second flight trajectory is provided to the background measurement area. Accordingly, the location of the background measurement area is arranged to define the location of the second flight trajectory or the vertical plane.

The second flight trajectory may be similar as the first flight trajectory.

In some embodiments, the second flight trajectory is similar one of the above disclosed first flight trajectories.

In some embodiments, the method comprises determining position of the tracer gas source, the tracer gas source being a stationary tracer gas source.

In some other embodiments, the method comprises determining position of the tracer gas source, the tracer gas source being a mobile tracer gas source.

In some embodiments, the tracer gas source is provided with tracer gas source positioning unit configured to determine the position of the tracer gas source.

In some embodiments, the step of determining the location of the emission measurement area in relation to the emission site comprises determining the location of the emission measurement area based on the determined position of the tracer gas source and the determined wind direction, or based on the determined position of the tracer gas source and the determined wind direction and wind speed.

In some other embodiments, the step of determining the location of the emission measurement area in relation to the emission site comprises determining location of the emission site, and determining the location of the emission measurement area based on the determined location of the emission site and the determined wind direction, or based on the determined location of the emission site and the determined wind direction and wind speed.

Accordingly, the location of the emission measurement area may be determined based on the location of the tracer gas source or the location of the emission site, and the determined location of the emission site and the determined wind direction and wind speed.

Thus, the direction of the emission measurement area in relation to the tracer gas source and/or the emission site may be determined. Further, distance of the emission measurement area from the tracer gas source and/or the emission site may be determined.

In some embodiment, the distance of the emission measurement area or the first flight directory from the tracer gas source and/or from the emission site may be predetermined. Thus, the distance of the emission measurement area may be defined to be constant in different measurements and emission sites.

In some embodiments, the step of determining the location of the background measurement area in relation to the emission site comprises determining the location of the background measurement area based on the determined position of the tracer gas source and the determined wind direction, or based on the determined position of the tracer gas source and the determined wind direction and wind speed.

In some other embodiments, the step of determining the location of the background measurement area in relation to the emission site comprises determining location of the emission site, and determining the location of the background measurement area based on the determined location of the emission site and the determined wind direction, or based on the determined location of the emission site and the determined wind direction and wind speed.

Accordingly, the location of the background measurement area may be determined based on the location of the tracer gas source or the location of the emission site, and the determined location of the emission site and the determined wind direction and wind speed.

Thus, the direction of the background measurement area in relation to the tracer gas source and/or the emission site may be determined. Further, distance of the background measurement area from the tracer gas source and/or the emission site may be determined.

In some embodiment, the distance of the background measurement area or the first flight directory from the tracer gas source and/or from the emission site may be predetermined. Thus, the distance of the background measurement area may be defined to be constant in different measurements and emission sites.

In some embodiments, the step of determining the location of the emission measurement area in relation to the emission site comprises providing a predetermined distance value between the emission measurement area and the position of the tracer gas source for determining the location of the emission measurement area in relation to the emission site.

In some other embodiments, the step of determining the location of the emission measurement area in relation to the emission site comprises providing a predetermined distance value between the emission measurement area and the location of the emission site for determining the location of the emission measurement area in relation to the emission site.

The predetermined distance of the emission measurement area enables providing comparable results in different emission sites and between different measurements occasions.

In some embodiments, the step of determining the location of the background measurement area in relation to the emission site comprises providing a predetermined distance value between the background measurement area and the position of the tracer gas source for determining the location of the background measurement area in relation to the emission site.

In some other embodiments, the step of determining the location of the background measurement area in relation to the emission site comprises providing a predetermined distance value between the background measurement area and the location of the emission site for determining the location of the background measurement area in relation to the emission site.

The predetermined distance of the background measurement area enables providing comparable results in different emission sites and between different measurements occasions.

In some embodiments, the step of controlling of the unmanned aerial vehicle comprises determining position of the unmanned aerial vehicle during the flight of the unmanned aerial vehicle for controlling the flight of unmanned aerial vehicle along the first flight trajectory, or along the second flight trajectory, or along the first and second flight trajectories.

In some embodiments, the method comprises determining the first flight trajectory, or the second flight trajectory, or the first and second flight trajectories before the flight of the unmanned aerial vehicle.

In some embodiments, the method comprises controlling the unmanned aerial vehicle with the control system to flight along the first flight trajectory in vicinity of the emission site, the first flight trajectory comprising at least two different;
measuring at a first altitude with the analyser unit of the unmanned aerial vehicle during the flight of the unmanned aerial vehicle along the first flight trajectory:
   first concentration of at least one airborne emission species emitted from the emission site, and
   first concentration of the tracer gas species emitted from the tracer gas source

The method comprises determining first emission rate of the at least one airborne emission species from the emission site at the first altitude of the first flight trajectory based on:
the determined tracer gas mass flow rate of the tracer gas species released from the tracer gas source,
the first measured concentration of the tracer gas species emitted from the tracer gas source, and
the first measured concentration of the at least one airborne emission species emitted from the emission site.

The method further comprises measuring at a second altitude with the analyser unit of the unmanned aerial vehicle during the flight of the unmanned aerial vehicle along the first flight trajectory:
second concentration of at least one airborne emission species emitted from the emission site, and
second concentration of the tracer gas species emitted from the tracer gas source

The method further comprises determining second emission rate of the at least one airborne emission species from the emission site at the second altitude of the first flight trajectory based on:
the determined tracer gas mass flow rate of the tracer gas species released from the tracer gas source,
the second measured concentration of the tracer gas species emitted from the tracer gas source, and
the second measured concentration of the at least one airborne emission species emitted from the emission site.

The method may be further carried out in a third altitude or number of successive altitudes.

The first altitude, the second altitude, and the possible third or successive altitudes may each comprise one constant altitude value when the unmanned aerial vehicle is moving substantially horizontally. Alternatively, the first altitude, the second altitude, and the possible third or successive altitudes may each be an average altitude of the unmanned aerial vehicle during the measuring or the sampling time or measurement interval.

In some embodiments, the method of present invention is carried out with a system as disclosed below.

The present invention is further based on an idea of providing a system configured to measure emissions rate of airborne emission species from an emission site, the emission site emitting one or more airborne emission species. The system comprises:
a tracer gas source configured to release tracer gas, the tracer gas source being arranged to the emission site and comprising a flow controlling device configured to determine tracer gas mass flow rate of the tracer gas from the tracer gas source:
an unmanned aerial vehicle comprising:
   a control system for controlling flight of the unmanned aerial vehicle, the control system being configured to control the unmanned aerial vehicle to flight along a first flight trajectory in vicinity of the emission site, the first flight trajectory comprising different altitudes, and
   an analyser unit provided to the unmanned aerial vehicle, the analyser unit being configured to measure at different altitudes concentration of at least one airborne emission species emitted from the emission site and concentration the tracer gas species emitted from the tracer gas source during the flight of the unmanned aerial vehicle along the first flight trajectory; and
processing unit configured to calculate emission rate of the at least one airborne emission species from the emission site at the different altitudes of the first flight trajectory based on:
   the determined tracer gas mass flow rate of the tracer gas species released from the tracer gas source,
   measured concentration of the tracer gas species emitted from the tracer gas source, and
   measured concentration of the at least one airborne emission species emitted from the emission site.

In some embodiments, the emission site is an industrial emission site having one or more emission sources,

In other embodiments, the emission site is an offshore emission site having one or more emission sources.

In some further embodiments, the emission site is a landfill emission site.

In some further embodiments, the emission site is a natural emission site, such as swamp, forest, sea, lake or the like.

In some further embodiments, the emission site is an agricultural emission site, such as a farm, a field, a barn or the like.

In some embodiments, the system comprises a wind sensor configured to determine wind direction, or wind direction and wind speed in the emission site or in the vicinity of the emission site.

The wind sensor may be provided to the emission site or to the unmanned aerial vehicle.

The wind sensor may also be an external weather service. The wind direction or wind direction and speed are received from the external weather service.

In some embodiments, the unmanned aerial vehicle is provided with a first positioning system configured to generate vehicle positioning data defining position of the unmanned aerial vehicle. The vehicle positioning data representing the location of the unmanned aerial vehicle during the flight.

In some embodiments, the tracer gas source is provided with a second positioning system configured to generate tracer positioning data defining position of the tracer gas source. The tracer positioning data representing the location of the tracer gas source.

In some embodiments, the tracer gas source is arranged to a stationary source platform.

In some other embodiments, the tracer gas source is arranged to a stationary source base, the stationary source platform comprises a longitudinal post extending in vertical direction and the tracer gas source is arranged to the longitudinal post at an elevated position.

In some further embodiments, the tracer gas source is arranged to a movable source platform.

In some yet further embodiments, the tracer gas source is arranged to a secondary unmanned aerial vehicle.

In some embodiments, the system comprises a display device configured to display on a map:
measured concentration tracer gas species emitted from the tracer gas source,
measured concentration of the at least one airborne emission species emitted from the emission site, and
position of the unmanned aerial vehicle.

In some embodiments, the system is configured to carry out the method as disclosed above.

An advantage of the invention is that the method and system of the present invention enable determining emission rate of an emission site in an efficient and accurate manner without restrictions caused by direction of wind. Furthermore, the method and system enable determining the emission rate at different altitudes such that most suitable measurement point or location is utilized in all weather and wind conditions. The present invention further enables carrying out measurements and determining the emission rate at corresponding height or location of the emission sources emitting the airborne emission species. Furthermore, the present invention enables determining emission rate of emission sites which are difficult or impossible to reach with conventional measurement equipment.

### BRIEF DESCRIPTION OF THE DRAWINGS

The invention is described in detail by means of specific embodiments with reference to the enclosed drawings, in which

FIGS. 1 to 3 show schematically one embodiment of an emission measurement system according to the present invention;

FIGS. 4 and 5 show schematically another embodiment of an emission measurement system according to the present invention;

FIG. 6 shows schematically yet another embodiment of an emission measurement system according to the present invention;

FIGS. 7 to 13 show schematically measuring emissions with an unmanned aerial vehicle; and

FIGS. 14 to 16 show schematically tracer gas arrangements for the emission measurement system according to the present invention.

### DETAILED DESCRIPTION OF THE INVENTION

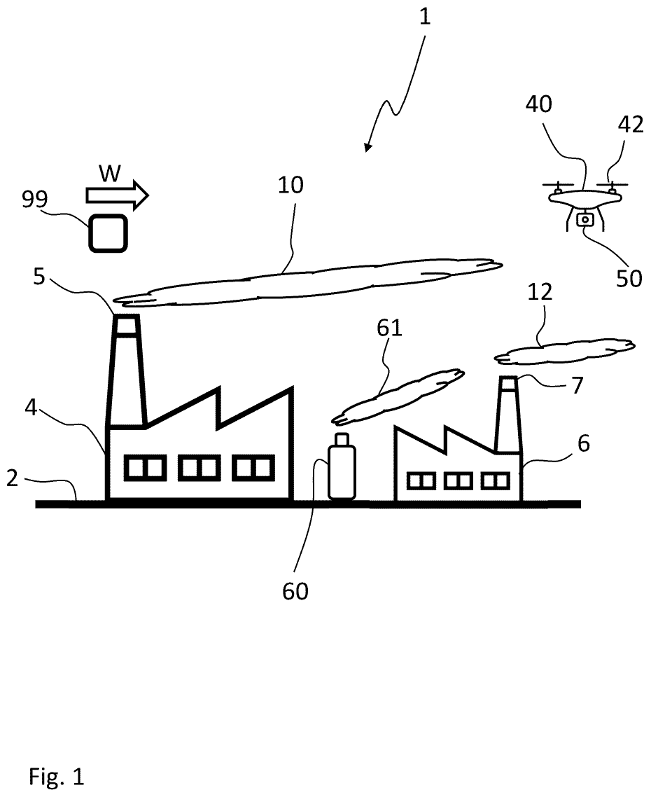

FIG. 1 shows an industrial emission site 1 having two industrial buildings 4 and 6. A first industrial building 4 comprises a first emission source 5 emitting one or more airborne emission species 10. The second industrial building 6 comprises a second emission source 7 emitting one or more airborne emission species 12.

In the embodiment of FIG. 1, the emission sources 5, 7 are chimneys. However, the emission sources 5, 7 may be any kind of industrial emission sources, including industrial processes and components like valves, flanges, pipes, pump, compressors and other devices. The emission sources may also be gas, liquid, sludge, solid material storages and/or treatment facilities, such as wastewater treatment pools and solid material storage piles.

The airborne emission species 10, 12 may be gaseous emission species, particulate emission species, aerosol emission species, flue gases or the like airborne emission species.

The emission sources 5, 7 are at heights from the ground 2.

A system according to the present invention comprises a tracer gas source 60 located or provided to the emission site 1.

The tracer gas source 60 comprises gas container containing tracer gas species.

The tracer gas source 60 is configured to release tracer gas species 61 from the gas container.

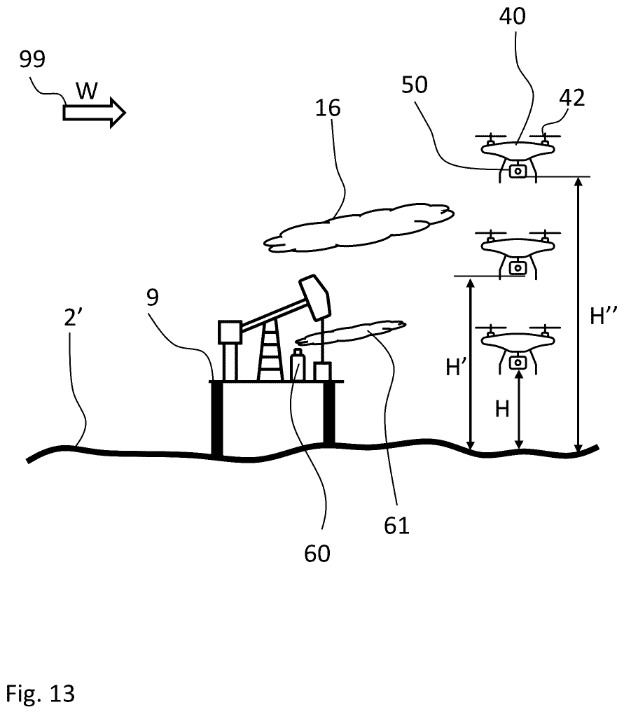

In the method and system of the present invention, the tracer gas source 60 is configured to control the release and tracer gas mass flow rate of the tracer gas species. The tracer gas source 60 comprises a flow controlling device 62 configured to determine tracer gas mass flow rate of the tracer gas 61 from the tracer gas source 60, as shown in FIGS. 13, 14 and 15.

The flow controlling device 62 may comprise a pressure or flow regulator or mass flow valve and a flow meter for determining and measuring the mass flow rate of the released tracer gas species 61.

In the method the tracer gas source 60 is configured to release a constant tracer gas mass flow rate of tracer gas species 61 during the method.

The tracer gas may be for example one of the following gases: N₂O, CO₂ and C₂H₂.

The tracer gas source 60 is preferably configured to sustain tracer gas mass flow rate between 0.1 kg/h and 10 kg/h with accuracy better than 5% for the duration of the measurement or flight of the unmanned aerial vehicle along a first flight trajectory.

The tracer gas source may further comprise a mass flow meter or the like flow meter configured to measure or record tracer gas mass flow rate of tracer gas species 61 released from the tracer gas source 60.

In some embodiments the tracer gas mass flow rate is predetermined and controlled with the flow controlling device 62.

In some other some embodiments the tracer gas mass flow rate is determined by the flow controlling device 62.

In some further embodiments, the tracer gas mass flow rate is controlled with the flow controlling device 62 and measured or recorded with the flow meter.

The system further comprises an unmanned aerial vehicle 40. The unmanned aerial vehicle 40 may be any kind unmanned aerial vehicle.

In some embodiments the unmanned aerial vehicle 40 is a fixed-wing vehicle, rotor operated vehicle, such as quadcopter or the like.

The unmanned aerial vehicle 40 is remotely controllable or automatically or autonomously controllable unmanned aerial vehicle 40. The unmanned aerial vehicle 40 may also be manually controllable.

The unmanned aerial vehicle 40 comprises a control system 42 configured to control the unmanned aerial vehicle 40 during flight. The control system 42 is preferably an electronic control system.

The control system 42 is configured to enable autonomous or manual control of the unmanned aerial vehicle 40.

The unmanned aerial vehicle 40 or the control system 42 thereof comprises propulsion device(s) for moving the unmanned aerial vehicle 40.

In some embodiments, the propulsion device(s) are part of the control system 42 or operatively connected to the control system 42. Accordingly, the control system 42 is configured to control the propulsion device(s) for controlling the unmanned aerial vehicle 40.

The unmanned aerial vehicle 40 may also comprise movable guiding elements (not shown) for controlling the movement of the unmanned aerial vehicle 40. In some embodiments, the guiding elements are part of the control system 42 or operatively connected to the control system 42.

The unmanned aerial vehicle 40 is further provided with an analyser unit 50 the analyser unit 50 is configured to concentrations of airborne species, gases, particulates or the like during the flight of the unmanned aerial vehicle 40.

The analyser unit 50 may comprise any kind of analyser configured to measure concentration airborne species.

The analyser unit 50 is configured intake ambient air and carry out analysis of the ambient air received in the analyser unit 50.

The analyser unit 50 may be a separate analyser unit releasably connected to the unmanned aerial vehicle 40. Alternatively, the analyser unit 50 is fixedly provided to the unmanned aerial vehicle 40.

The analyser unit 50 comprises a sampling port (not shown) open to ambient air for intaking or receiving ambient air into the analyser unit 50 for analysis.

The analyser unit 50 further comprises an analyser 52 configured to measure concentration of airborne species in the ambient air received via the sampling port. Analysers 52 carrying out measurements for the received ambient air are so called sniffer type analysers. These analysers 52 may be any kind of analysers. In some embodiments, comprises one or more sensors, such as laser spectroscopy sensor, electrochemical sensor, nondispersive infrared sensor, photoionization sensor or the like.

In some alternative embodiments, the analyser unit 50 comprises one or more remote sensing sensors, such as remote sensing laser sensors, as analysers 52. The remote sensing sensor 52 is configured to carry out measurement of airborne species remotely without receiving ambient air into the sensor. For example, a remote sensing laser sensor is configured to register all gas molecules and/or particles in the light path of the laser beam. In this kind of sensor, the laser beam may be directed to point downwards from the unmanned aerial vehicle 40.

The remote sensing sensor(s) may be provided instead of the sniffer type sensors and in addition to the sniffer type sensors.

It should be noted, that the present invention is not restricted to any particular type of analyser 52 or sensor.

In some embodiments, the analyser 52 of the analyser unit 50 preferably has temporal sampling time with a response of 10 seconds or less.

The unmanned aerial vehicle 40 is further provided with a first positioning system configured to generate vehicle positioning data defining position of the unmanned aerial vehicle 40 during the flight of the unmanned aerial vehicle 40.

The first positioning system is preferably a global positioning system (GPS) or some other global navigation satellite system (GNSS). The first positioning system comprises GPS receiver or GNSS receiver configured to receive satellite signals for generating position data of the unmanned aerial vehicle 40 for determining the position of the unmanned aerial vehicle 40.

The first positioning system may also be positioning system utilizing local radio beacons, 5G or beyond stations or local area network such as WiFi.

In preferable embodiments, temporal sampling time or refresh interval of the first positioning system is at least 1 second during flight of the unmanned aerial vehicle 40. The temporal sampling time means time interval between successive determination of the position of the unmanned aerial vehicle 40 during flight.

The first positioning system is provided to the analyser unit 50. Alternatively, the first positioning system is provided to directly to the unmanned aerial vehicle 40.

In some embodiments, the tracer gas source 60 is further provided with a second positioning system configured to generate tracer positioning data defining position of the tracer gas source 60.

The second positioning system is preferably a global positioning system (GPS) or some other global navigation satellite system (GNSS). The second positioning system comprises GPS receiver or GNSS receiver configured to receive satellite signals for generating position data of the tracer gas source 60 for determining the position of the tracer gas source 60.

The second positioning system may also be positioning system utilizing local radio beacons, 5G or beyond stations or local area network such as WiFi.

In some embodiments, temporal sampling time or refresh interval of the second positioning system is at least 1 second. In some other embodiments, the temporal sampling time of the second positioning system is between 1 to 30 seconds. The temporal sampling time means time interval between successive determination of the position of the tracer gas source 60.

In some further embodiments, the position of the tracer gas source 60 is determined before flight of the unmanned aerial vehicle 40. Thus, the position of the tracer gas source 60 is not refreshed during the flight of the unmanned aerial vehicle 40.

The system may further comprise a wind sensor 99 configured to determine wind direction W, or wind direction W and wind speed in the emission site 1 or in the vicinity of the emission site 1. The wind sensor 99 may be any known kind of wind sensor.

In some embodiments, the wind sensor 99 is provided as a separate sensor to the emission site.

In some other embodiments, the wind sensor 99 is provided to the unmanned aerial vehicle 40.

In some further embodiments, the wind sensor 99 is provided in connection with the tracer gas source 60.

In some embodiments the wind sensor 99 is omitted, and the system is configured to receive wind data from an external weather service (not shown). The wind data comprises direction W or wind direction W and wind speed information relating to the emission site 1 or vicinity thereof, or the location of the emission site 1. Accordingly, the external weather service may operate as the wind sensor 99.

In some further embodiments, the wind direction information inputted to the system. Thus, the wind direction may be based on visual inspection at the emission site 1.

All the above mentioned aspects of the system of the present invention may be combined in any possible manner depending on the application of the system, the emission site 1 and weather circumstances.

FIGS. 1 to 3 show one embodiment in which the emission site 1 is the industrial emission site 1. The tracer gas source 60 is arranged to the emission site 1 and configured to release tracer gas species 61 from a tracer gas source 60 at a determined tracer gas mass flow rate.

The emission site 1 comprises two emission sources 5, 7 emitting airborne emission species 10, 12, respectively. In this embodiment the emission sources 5, 7 and point-like emission sources.

A wind is directed to a wind direction W. The wind direction moves the airborne emission species 10, 12 and the released tracer gas species 61 along the wind direction W downwind direction of the emission site 1. The wind direction W may be determined with the wind sensor 99. The wind sensor 99 may also determine wind speed.

The unmanned aerial vehicle 40 is provided with the analyser unit 50. The analyser unit 50 is configured to measure concentration of at least one airborne emission species 10, 12 emitted from the emission site 1, and concentration the tracer gas species 61 emitted from the tracer gas source 60 at different altitudes.

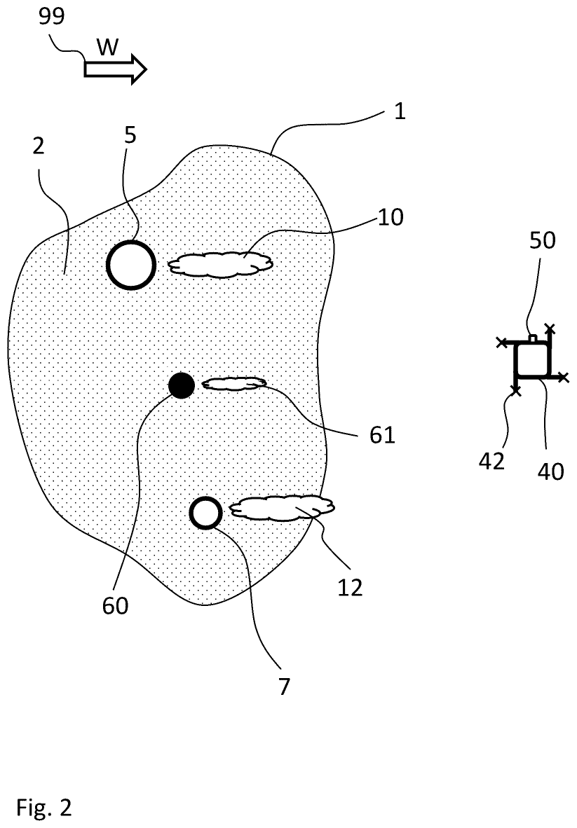

The location for carrying out the measurement is dependent on the wind direction. Therefore, the method comprises determining location of an emission measurement area in relation to the emission site 1 based on the determined wind direction W or based on the determined wind direction W and wind speed. The location of the emission measurement area is in downwind direction from the emission site 1, as shown in FIG. 2.

For carrying out the measurements, controlling the unmanned aerial vehicle 40 with the control system 42 to flight along a first flight trajectory 44 in vicinity of the emission site 1 in the emission measurement area.

The airborne emission species 10, 12 and the tracer gas species are mixed in the atmosphere and spread to different altitudes in the atmosphere. Therefore, for achieving reliable measurement results, the measurements needs to be carried out at different altitudes.

The first flight trajectory 44 comprises different altitudes for carrying out the measurements during the flight of the unmanned aerial vehicle 40 at different altitudes along the first flight trajectory.

The aim of the method of the present invention is to determine amount or quantity of airborne emission species 10, 12 from the emission site 1. Thus, the method comprises determining or calculating emission rate of the at least one airborne emission species 10, 12 from the emission site 1 at the different altitudes of the first flight trajectory 44 based on the determined tracer gas mass flow rate of the tracer gas species 61 released from the tracer gas source 60, measured concentration tracer gas species 61 emitted from the tracer gas source 60, and measured concentration of the at least one airborne emission species 10, 12 emitted from the emission site 1.

In the method and system of the present invention, there may be provided one or more, or two or more tracer gas sources to the emission site 1 or in the vicinity thereof.

The two or more tracer gas sources may be positioned at a distance from each other so that the area of the emission site may be covered.

When the emission site 1 comprises two or more separate emission sources, the two or more tracer gas sources may be positioned in connected or near the two or more separate emission sources. Accordingly, one tracer gas source may be arranged in connection with each or at least some of the separate emission sources.

The two more tracer gas sources are configured to release same or different tracer gas species.

Utilizing different tracer gas species in connection with different separate emission sources in the emission site enables detecting or measuring emission rates from different separate emission sources.

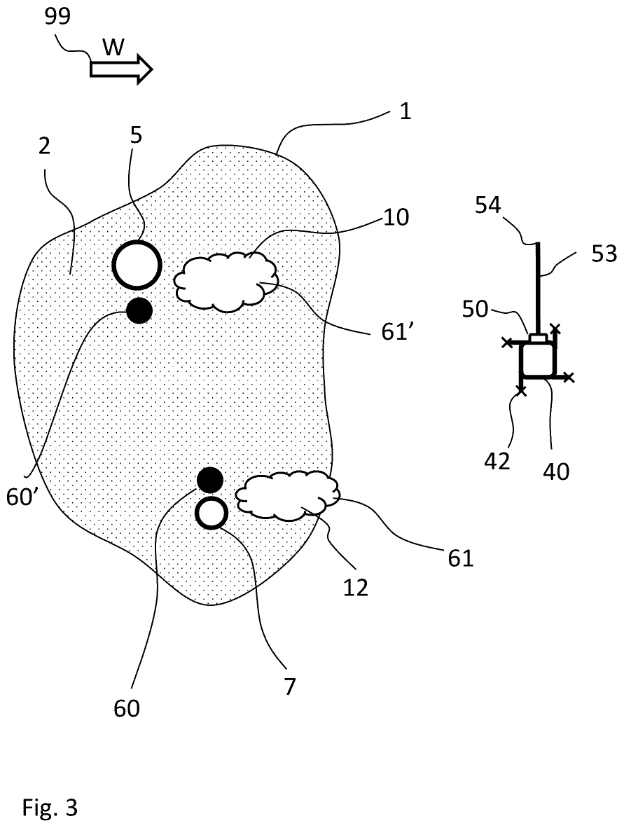

FIG. 3 shows an embodiment in which the emission site 1 comprises a first emission sources 5 emitting first airborne emission species 10. A first tracer gas source 60′ is arranged in connection with the first emission source 5 in the emission site 1 and releasing first tracer species 61′. The emission site 1 also comprises a second emission sources 7 emitting second airborne emission species 12. A second tracer gas source 60 is arranged in connection with the second emission source 7 in the emission site 1 and releasing second tracer species 61′.

In this embodiment, the method comprises measuring at different altitudes with the analyser unit 50 of the unmanned aerial vehicle 40 during the flight of the unmanned aerial vehicle 40 along the first flight trajectory 44:
concentration of the first airborne emission species 10 emitted from the first emission source 5,
concentration of the second airborne emission species 12 emitted from the second emission source 7
concentration of the first and second tracer gas species 61, 61′ emitted from the first and second tracer gas source 60, 60′.

The method further comprises determining emission rate of the first and second airborne emission species 10, 12 from the emission site 1 at the different altitudes of the first flight trajectory 44 based on:
the determined tracer gas mass flow rates of the first and second tracer gas species 61, 61′released from the first and second tracer gas source 60, 60′, respectively,
measured concentration of the first and second tracer gas species 61, 61′emitted from the first and second tracer gas source 60, 60′, and
measured concentration of the first and second airborne emission species 10, 12 emitted from the emission site 1.

As shown in FIG. 3, the analyser unit 50 is provided with an inlet tube 53 extending away from the unmanned aerial vehicle 50. The inlet tube 53 is connected to the analyser 52 and arranged conduct ambient air to the analyser 52. The inlet tube 53 comprises a sampling port 54 or sampling opening open to the ambient atmosphere for receiving ambient air. The sampling port 54 is provided to the distal end of the inlet tube 53. The sampling port 54 is arranged at distance away from the unmanned aerial vehicle 40 such that the unmanned aerial vehicle 40 or the rotors 42 do not disturb or affect the ambient air receiving the analyser 52.

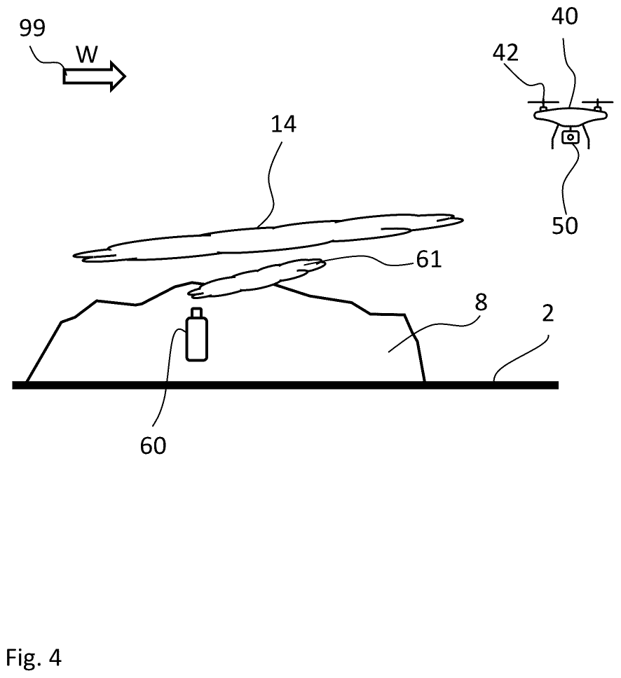

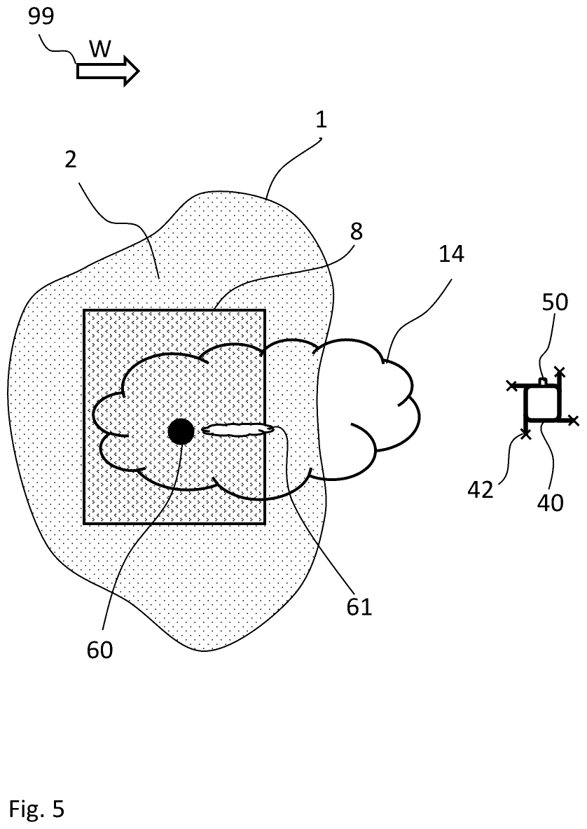

FIGS. 4 and 5 show another embodiment in which the emission site 1 is a landfill 8 emitting one or more airborne emission species 14 such as methane. In this embodiment, the landfill 8 forms the emission source. The emission source is an area emission source.

In this embodiment the tracer gas source 60 is provided to the landfill 8 and configured to release tracer gas species 61 from the tracer gas source 60 at a determined tracer gas mass flow rate.

The emission measurement area is defined in the same manner as in the embodiment FIGS. 1 to 3. Furthermore, the unmanned aerial vehicle 40 is controlled with the control system 42 to flight along the first flight trajectory 44 in vicinity of the emission site 1 in the emission measurement area. The analyser unit 50 is configured to measuring concentration of at least one airborne emission species 14 emitted from the emission site 1, and concentration the tracer gas species 61 emitted from the tracer gas source 60 at different altitudes during the flight of the unmanned aerial vehicle 40 along the first flight trajectory 44.

The unmanned aerial vehicle 40 may be controlled in the same manner and along a similar first flight trajectory as in the embodiment of FIGS. 1 and 2.

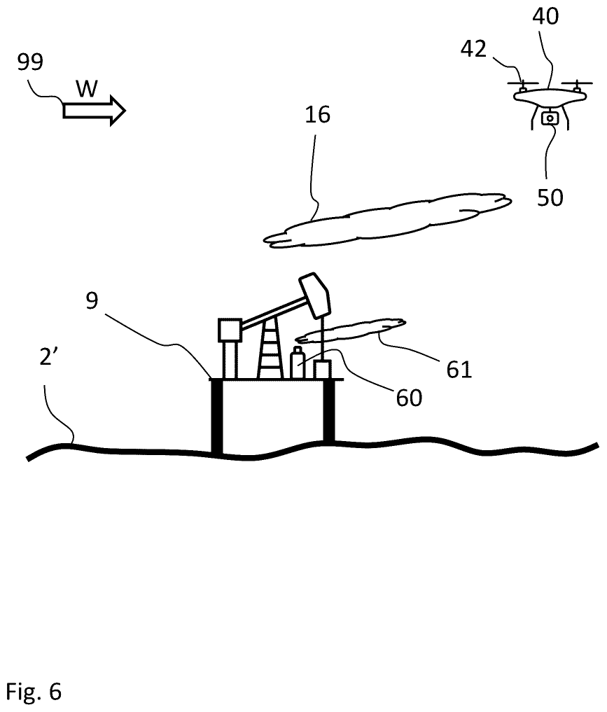

FIG. 6 shows a further embodiment in which the emission site is an offshore emission site, and specifically an offshore oil rig 9 emitting airborne emission species 16.

In this embodiment the tracer gas source 60 is provided to the offshore emission site 9 and configured to release tracer gas species 61 from the tracer gas source 60 at a determined tracer gas mass flow rate.

The emission measurement area is defined in the same manner as in the embodiment FIGS. 1 and 2 based on the wind direction W or wind direction W and wind speed. Furthermore, the unmanned aerial vehicle 40 is controlled with the control system 42 to flight along the first flight trajectory 44 in vicinity of the emission site 1 in the emission measurement area. The analyser unit 50 is configured to measuring concentration of at least one airborne emission species 16 emitted from the emission site 1, and concentration the tracer gas species 61 emitted from the tracer gas source 60 at different altitudes during the flight of the unmanned aerial vehicle 40 along the first flight trajectory 44.

The unmanned aerial vehicle 40 may be controlled in the same manner and along a similar first flight trajectory as in the embodiment of FIGS. 1 and 2.

In the embodiments of FIGS. 4 to 6, the method also comprises determining or calculating emission rate of the at least one airborne emission species 14, 16 from the emission site 1 at the different altitudes of the first flight trajectory 44 based on the determined tracer gas mass flow rate of the tracer gas species 61 released from the tracer gas source 60, measured concentration tracer gas species 61 emitted from the tracer gas source 60, and measured concentration of the at least one airborne emission species 14, 16 emitted from the emission site 1.

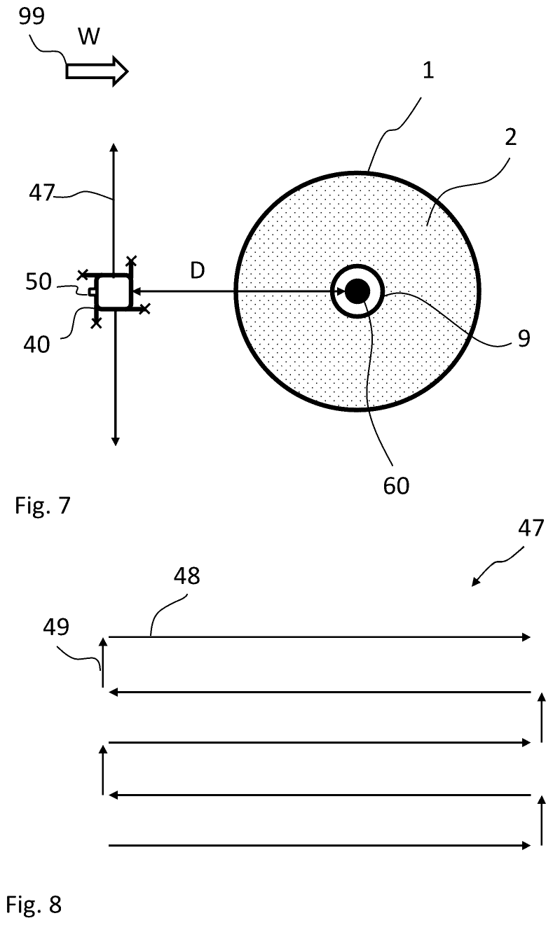

The offshore oil rig 9 and embodiment of FIG. 6 is used as an example of the method in FIGS. 7 to 13.

In order to provide accurate measurement results, background airborne emission species needs to be taken into account and deducted form the measurement results measured in the emission measurement are along the first flight trajectory.

Therefore, in some embodiments the method comprises measuring background airborne emission species, as shown in FIGS. 7 and 8.

Thus, the method comprises determining location of a background measurement area in relation to the emission site 1 based on the determined wind direction W or based on the determined wind direction W and wind speed. The location of the background measurement area is in upwind direction from the emission site 1.

Then, the unmanned aerial vehicle 40 is controlled with the control system 42 to flight along a second flight trajectory 47 in the vicinity of the emission site 1 in the background measurement area, as shown in FIGS. 7 and 8. During the flight background emissions are measured with the analyser unit 50 of the unmanned aerial vehicle 40 during the flight of the unmanned aerial vehicle 40 along the first flight trajectory 44 at different altitudes in the background measurement area by measuring concentration of at least one background airborne emission species in the background emission area.

The measured concentration of at least one background airborne emission species is deducted from the measured concentration airborne emission species and tracer gas species in the emission measurement area.

In the embodiment of FIGS. 7 and 8, the second flight trajectory 47 comprises horizontal flight paths 48. The horizontal flight paths 48 are at different altitudes, and the measuring of the concentration of the measuring the background airborne emission species in the background measurement area is carried along the horizontal flight paths 48 at different altitudes during the flight of the unmanned aerial vehicle 40.

The horizontal flight paths 48 may be linear transects or curved flight paths.

Accordingly, the unmanned aerial vehicle is controlled to move successively along the horizontal flight paths 48 of the second flight trajectory 47 and carry out the measurements in at least two of the horizontal flight paths 48 at different altitudes with the analyser unit 50 for measuring the background airborne emission species.

The second flight trajectory 47 also comprises vertical flight paths 49 for moving the unmanned aerial vehicle 40 from one horizontal flight path 48 to another.

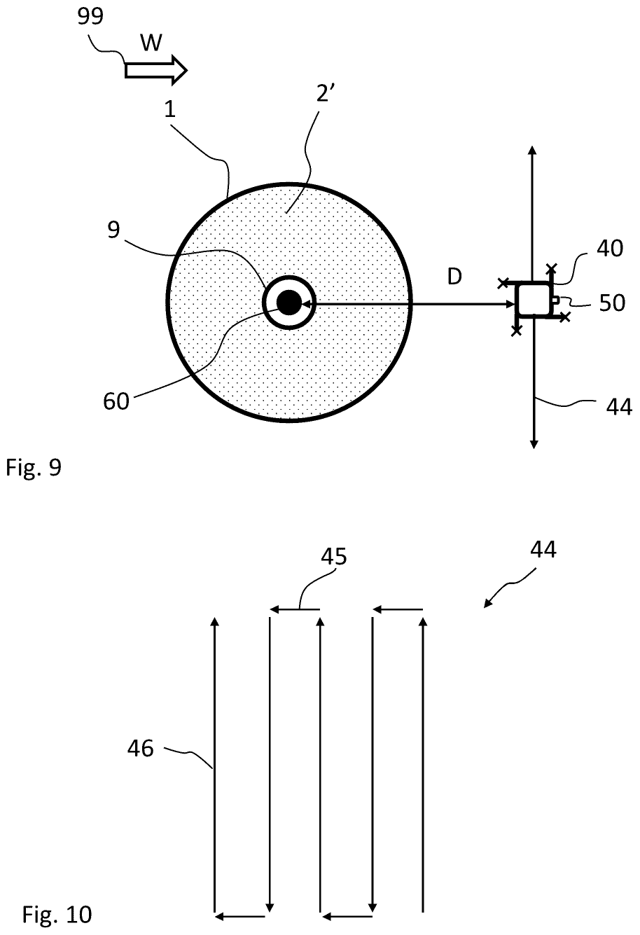

In the embodiment of FIGS. 9 and 10, the first flight trajectory 44 comprises vertical flight paths 46. The vertical flight paths 46 are in vertical direction and extend along different altitudes, and the measuring of the concentration of the at least one airborne emission species 16 emitted from the emission site 1 and of the concentration the tracer gas species 61 emitted from the tracer gas source 60 in the emission measurement area is carried along the vertical flight paths 46 at different altitudes during the flight of the unmanned aerial vehicle 40.

The vertical flight paths 45 may be linear transects or curved flight paths.

Accordingly, the unmanned aerial vehicle is controlled to move successively along the vertical flight paths 46 of the first flight trajectory 44 and carry out the measurements in at least two of the vertical fly paths 46 at different altitudes with the analyser unit 50 for measuring the concentration of the at least one airborne emission species 16 emitted from the emission site 1 and of the concentration the tracer gas species 61 emitted from the tracer gas source 60.

The first flight trajectory 44 also comprises horizontal flight paths 45 for moving the unmanned aerial vehicle 40 from one vertical flight path 46 to another.

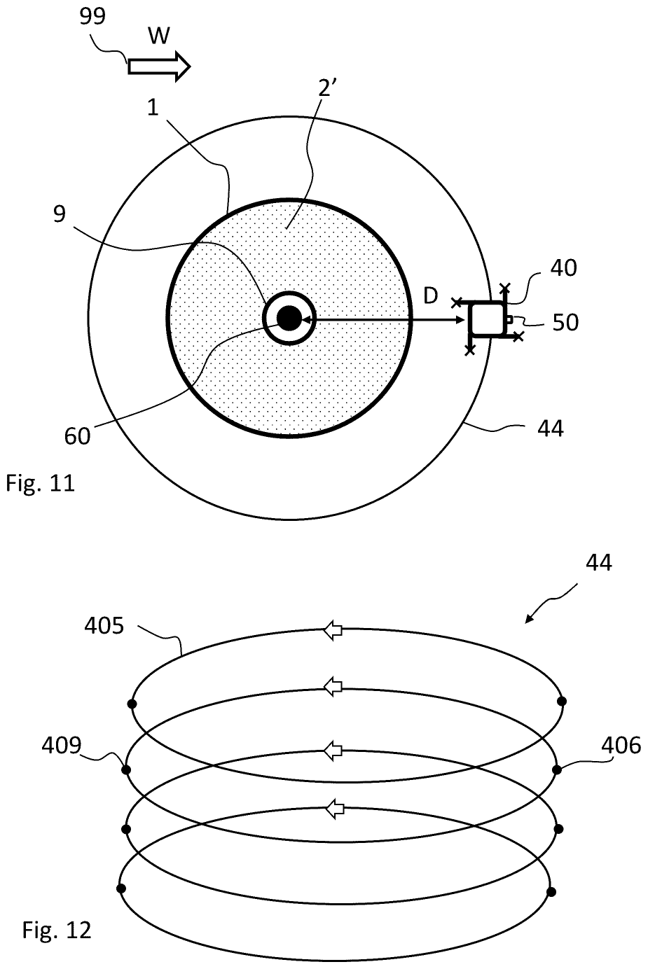

FIGS. 11 and 12 show a different example of the first flight trajectory 44. The first flight trajectory 44 comprises circumferential flight paths 405 surrounding the emission site 1 and the tracer gas source 60. The circumferential flight paths 405 are at different altitudes. The measuring of the concentration of the at least one airborne emission species 16 emitted from the emission site 1 and of the concentration the tracer gas species 61 emitted from the tracer gas source 60 in the emission measurement area is carried along the circumferential flight paths 405 at different altitudes during the flight of the unmanned aerial vehicle 40.

The circumferential flight paths 405 may be circular, elliptical polygonal or any other shape flight paths. The circumferential flight paths 405 are at different altitudes H, H′, H″, as shown in FIG. 12.

As shown in FIGS. 7, 8, and 11 the background measurement area and the emission measurement area are arranged to a distance D from the emission site 1, or the emission source 9 or the tracer gas source 60.

Thus, the method comprises determining the location of the emission measurement area and/or the background measurement area based on the determined position of the tracer gas source 60 and the determined wind direction W, or based on the determined position of the tracer gas source 60 and the determined wind direction W and wind speed.

The unmanned aerial vehicle 40 is provided with the first positioning system 42 generating vehicle positioning data and the tracer gas source 60 is provided the second positioning system generating tracer positioning data.

The vehicle positioning data and the tracer positioning data are utilized to control the unmanned aerial vehicle 40 along the first and/or second flight trajectory 45, 47 in the determined location of the emission measurement area and/or the background measurement area in relation tracer gas source 60.

In some embodiments, a distance value D between the emission measurement area and/or the background measurement area and the position of the tracer gas source 60 is determined. Thus, the first and/or second flight trajectory is configured at the predetermined distance value D from the tracer gas source 60.

The predetermined distance value may be a fixed value. Alternatively, the predetermined distance D may be determined for example based on the wind direction W, or based on the wind direction W and wind speed.

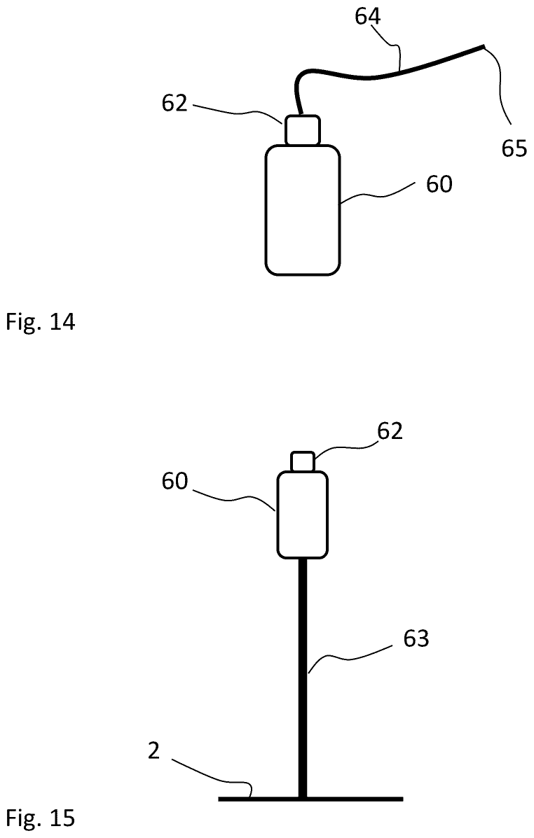

FIG. 14 shows schematically the tracer gas source 60 with the flow tracer gas container and tracer gas flow controlling device 62. The tracer gas source 60 of FIG. 14 is arranged as a stationary tracer gas source 60.

The flow controlling device 62 may also comprise flow meter.

In the embodiment of FIG. 14, the tracer gas source 60 comprises an outlet tube 64 extending from the flow tracer gas container and/or tracer gas flow controlling device 62. The outlet tube 64 comprises an outlet opening 65 at the distant end of the outlet tube 64. The tracer gas species are released to the atmosphere from the outlet opening 65.

In the context of this application the location or position of the tracer gas sources 60 means the location of the outlet opening 65 from which the tracer gas species are released to the atmosphere.

FIG. 15 shows an alternative embodiment, in which the tracer gas source 60 is arranged to a stationary source base 63. The stationary source platform 63 comprises a longitudinal post extending in vertical direction and the tracer gas source 60 is arranged to the longitudinal post 63 at an elevated position. Thus, the tracer gas may be released at height close to or corresponding the emission sources.

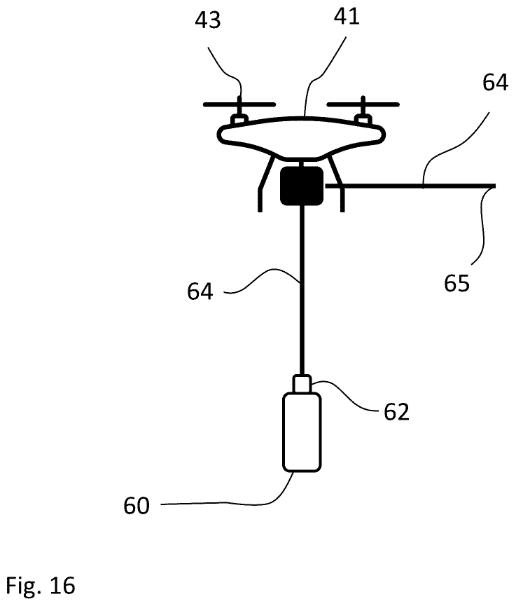

FIG. 16 shows a further alternative embodiment, in which the tracer gas source 60 is provided in connection with a secondary unmanned aerial vehicle 41. Thus, the tracer gas source 60 of FIG. 14 is provided as movable tracer gas source.

The outlet tube 64 is connected to the secondary unmanned aerial vehicle 41 such that the tracer gas species are released via the outlet opening 65. The location or position of the outlet opening 65 may be moved by moving the secondary unmanned aerial vehicle 41. Thus, a movable tracer gas source is provided. The tracer gas container may be provided to ground or alternatively it may be provided to the secondary unmanned aerial vehicle 41.

The invention has been described above with reference to the examples shown in the figures. However, the invention is in no way restricted to the above examples but may vary within the scope of the claims.

1. A method for measuring emission rates of airborne emission species from an emission site the emission site emitting one or more airborne emission species, the method comprising:
releasing tracer gas species from a tracer gas source at a determined tracer gas mass flow rate, the tracer gas source being provided to the emission site or in the vicinity thereof,
providing an unmanned aerial vehicle comprising:
a control system for controlling flight of the unmanned aerial vehicle, and
an analyser unit provided to the unmanned aerial vehicle;
controlling the unmanned aerial vehicle with the control system to flight along a first flight trajectory in vicinity of the emission site the first flight trajectory comprising different altitudes;
measuring at different altitudes with the analyser unit of the unmanned aerial vehicle during the flight of the unmanned aerial vehicle along the first flight trajectory:
concentration of at least one airborne emission species emitted from the emission site, and
concentration of the tracer gas species emitted from the tracer gas source; and
determining emission rate of the at least one airborne emission species from the emission site at the different altitudes of the first flight trajectory based on:
the determined tracer gas mass flow rate of the tracer gas species released from the tracer gas source,
measured concentration of the tracer gas species emitted from the tracer gas source, and
measured concentration of the at least one airborne emission species emitted from the emission site.

2. The method according to claim 1, further comprising:
controlling the unmanned aerial vehicle with the control system along the first flight trajectory, wherein
the first flight trajectory comprises measurement intervals along the first flight trajectory at different altitudes, the measuring of the concentration of the at least one airborne emission species emitted from the emission site and of the concentration the tracer gas species emitted from the tracer gas source is carried out in the measurement intervals during the flight of the unmanned aerial vehicle; or
the measuring of the concentration of the at least one airborne emission species emitted from the emission site and measuring of the concentration the tracer gas species emitted from the tracer gas source is carried out continuously during the flight of the unmanned aerial vehicle along the first flight trajectory at different altitudes.

3. (canceled)

4. (canceled)

5. The method according to claim 1, further comprising:
determining wind direction in the emission site or in the vicinity of the emission site; or
determining wind direction and wind speed in the emission site or in the vicinity of the emission site.

6. The method according to claim 5, characterized in that the method further comprises:
determining location of an emission measurement area in relation to the emission site based on the determined wind direction or based on the determined wind direction and wind speed, the location of the emission measurement area being in downwind direction from the emission site,
controlling the unmanned aerial vehicle with the control system to flight along the first flight trajectory in the vicinity of the emission site in the emission measurement area, and
measuring emissions from the emission site with the analyser unit of the unmanned aerial vehicle during the flight of the unmanned aerial vehicle along the first flight trajectory at different altitudes in the emission measurement area by measuring:
concentration of at least one airborne emission species emitted from the emission site in the emission measurement area, and
concentration of the tracer gas species emitted from the tracer gas source in the emission measurement area.

7. The method according to claim 5, further comprising:
determining location of the background measurement area in relation to the emission site based on the determined wind direction or based on the determined wind direction and wind speed, the location of the background measurement area being in upwind direction from the emission site,
controlling the unmanned aerial vehicle with the control system to flight along the first flight trajectory in the vicinity of the emission site in the background measurement area, and
measuring background emissions with the analyser unit of the unmanned aerial vehicle during the flight of the unmanned aerial vehicle along the first flight trajectory at different altitudes in the background measurement area by measuring concentration of at least one background airborne emission species in the background emission area; or
determining location of the background measurement area in relation to the emission site based on the determined wind direction or based on the determined wind direction and wind speed, the location of the background measurement area being in upwind direction from the emission site,
controlling the unmanned aerial vehicle with the control system to flight along a second flight trajectory in the vicinity of the emission site in the background measurement area, the second flight trajectory comprising different altitudes, and
measuring background emissions with the analyser unit of the unmanned aerial vehicle during the flight of the unmanned aerial vehicle along the second flight trajectory at different altitudes in the background measurement area by measuring concentration of at least one background airborne emission species in the background emission area.

8. The method according to claim 1, further comprising:
determining position of the tracer gas source the tracer gas source being a stationary tracer gas source; or
determining position of the tracer gas source the tracer gas source being a mobile tracer gas source.

9. The method according to claim 7, wherein the step of determining the location of the emission measurement area in relation to the emission site comprises:
determining the location of the emission measurement area based on the determined position of the tracer gas source and the determined wind direction or based on the determined position of the tracer gas source and the determined wind direction and wind speed; or
determining location of the emission site, and
determining the location of the emission measurement area based on the determined location of the emission site and the determined wind direction, or based on the determined location of the emission site and the determined wind direction and wind speed.

10. The method according to claim 7, wherein the step of determining the location of the background measurement area in relation to the emission site (1) comprises:
determining the location of the background measurement area based on the determined position of the tracer gas source and the determined wind direction or based on the determined position of the tracer gas source and the determined wind direction and wind speed; or
determining location of the emission site, and
determining the location of the background measurement area based on the determined location of the emission site and the determined wind direction, or based on the determined location of the emission site and the determined wind direction and wind speed.

11. (canceled)

12. (canceled)

13. The method according to claim 1, wherein the step of controlling of the unmanned aerial vehicle comprises determining position of the unmanned aerial vehicle during the flight of the unmanned aerial vehicle for controlling the flight of unmanned aerial vehicle along the first flight trajectory, or along the second flight trajectory or along the first and second flight trajectories.

14. The method according to claim 1, wherein the method comprises determining the first flight trajectory, or the second flight trajectory, or the first and second flight trajectories before the flight of the unmanned aerial vehicle.

15. The method according to claim 1, characterized in that the method is carried out with a system configured to measure emissions rate of airborne emission species from an emission site, the emission site emitting one or more airborne emission species comprising:
a tracer gas source configured to release tracer gas, the tracer gas source being arranged to the emission site (or in vicinity thereof and comprising a flow controlling device configured to determine tracer gas mass flow rate of the tracer gas from the tracer gas source:
an unmanned aerial vehicle comprising:
a control system for controlling flight of the unmanned aerial vehicle, the control system being configured to control the unmanned aerial vehicle to flight along a first flight trajectory in vicinity of the emission site, the first flight trajectory comprising different altitudes, and
an analyser unit provided to the unmanned aerial vehicle, the analyser unit being configured to measure at different altitudes concentration of at least one airborne emission species emitted from the emission site and concentration the of tracer gas species emitted from the tracer gas source during the flight of the unmanned aerial vehicle along the first flight trajectory; and
a processing unit configured to calculate emission rate of the at least one airborne emission species from the emission site at the different altitudes of the first flight trajectory based on:
the determined tracer gas mass flow rate of the tracer gas species released from the tracer gas source,
measured concentration of the tracer gas species emitted from the tracer gas source, and
measured concentration of the at least one airborne emission species emitted from the emission site.

16. A system configured to measure emissions rate of airborne emission species from an emission site the emission site emitting one or more airborne emission species, the system comprising:
a tracer gas source configured to release tracer gas, the tracer gas source being arranged to the emission site or in vicinity thereof and comprising a flow controlling device configured to determine tracer gas mass flow rate of the tracer gas from the tracer gas source:
an unmanned aerial vehicle comprising:
a control system for controlling flight of the unmanned aerial vehicle, the control system being configured to control the unmanned aerial vehicle to flight along a first flight trajectory in vicinity of the emission site, the first flight trajectory comprising different altitudes, and
an analyser unit provided to the unmanned aerial vehicle, the analyser unit being configured to measure at different altitudes concentration of at least one airborne emission species emitted from the emission site and concentration the of tracer gas species emitted from the tracer gas source during the flight of the unmanned aerial vehicle along the first flight trajectory; and
a processing unit configured to calculate emission rate of the at least one airborne emission species from the emission site at the different altitudes of the first flight trajectory based on:
the determined tracer gas mass flow rate of the tracer gas species released from the tracer gas source,
measured concentration of the tracer gas species emitted from the tracer gas source, and
measured concentration of the at least one airborne emission species emitted from the emission site.

17. The system according to claim 16, wherein the emission site is:
an industrial emission site having one or more emission sources; or
an offshore emission site having one or more emission sources; or
a landfill emission site; or
a natural emission site; or
an agricultural emission site.

18. The system according to claim 16, further comprising a wind sensor configured to determine wind direction, or wind direction and wind speed in the emission site or in the vicinity of the emission site.

19. The system according to claim 16, wherein the unmanned aerial vehicle is provided with a first positioning system configured to generate vehicle positioning data defining position of the unmanned aerial vehicle.

20. The system according to claim 16, wherein the tracer gas source is provided with a second positioning system configured to generate tracer source positioning data defining position of the tracer gas source.

21. The system according to claim 16, wherein
the tracer gas source is arranged to a stationary source platform or
the tracer gas source is arranged to a stationary source base the stationary source platform comprises a longitudinal post extending in vertical direction and the tracer gas source is arranged to the longitudinal post at an elevated position; or
the tracer gas source is arranged to a movable source platform; or
the tracer gas source is arranged to a secondary unmanned aerial vehicle.

22. The system according to claim 19, further comprising a display device configured to display on a map:
measured concentration of the tracer gas species emitted from the tracer gas source,
measured concentration of the at least one airborne emission species emitted from the emission site, and
position of the unmanned aerial vehicle.

23. The system according to claim 16, characterized in that the system is configured to carry out a method comprising:
releasing tracer gas species from a tracer gas source at a determined tracer gas mass flow rate, the tracer gas source being provided to the emission site or in the vicinity thereof,
providing an unmanned aerial vehicle comprising:
a control system for controlling flight of the unmanned aerial vehicle, and
an analyser unit provided to the unmanned aerial vehicle;
controlling the unmanned aerial vehicle with the control system to flight along a first flight trajectory in vicinity of the emission site, the first flight trajectory comprising different altitudes;
measuring at different altitudes with the analyser unit of the unmanned aerial vehicle during the flight of the unmanned aerial vehicle along the first flight trajectory:
concentration of at least one airborne emission species emitted from the emission site, and
concentration of the tracer gas species emitted from the tracer gas source; and
determining emission rate of the at least one airborne emission species from the emission site at the different altitudes of the first flight trajectory based on:
the determined tracer gas mass flow rate of the tracer gas species released from the tracer gas source,
measured concentration of the tracer gas species emitted from the tracer gas source, and
measured concentration of the at least one airborne emission species emitted from the emission site.
